(19) United States
(12) Patent Application Publication
(10) Pub. No.: US 2011/0224549 A1
(43) Pub. Date: Sep. 15, 2011

(54) ULTRASONIC DIAGNOSTIC APPARATUS AND METHOD FOR GENERATING ULTRASONIC IMAGES

(75) Inventor: Takehiro Tsujita, Tokyo (JP)

(73) Assignee: HITACHI MEDICAL CORPORATION, Tokyo (JP)

(21) Appl. No.: 13/129,303

(22) PCT Filed: Nov. 10, 2009
(86) PCT No.: PCT/JP2009/069080

§ 371 Date: May 13, 2011

(30) Foreign Application Priority Data
Nov. 14, 2008 (JP) 2008-292488

(51) Int. Cl. A61B 8/00

(52) U.S. Cl. 600/443

(57) ABSTRACT

When a series of plural data processes are processed by different processors, the need to harmonize the processing cycles between the respective processors and adjust the processing time is obviated. The invention includes a data conversion section 20 that generates ultrasonic image data using RF data based on reflected echo signals received from a subject. The data conversion section 20 includes a plurality of processors 20a to 20h that perform a series of data processes related to an ultrasonic measurement mode and a buffer memory 20m that stores the processing results. The respective processors 20a to 20h perform processes that are assigned to them in accordance with a write request of their own processor, write the processing results in the buffer memory 20m, and read the processing results written in the buffer memory 20m in accordance with a read request of other processors.

### TECHNICAL FIELD

The invention relates to an ultrasonic diagnostic apparatus and a method for generating ultrasonic images, and more particularly, to an improvement of a data conversion section that performs an arithmetic process of converting reflected echo signals into ultrasonic images.

### BACKGROUND ART

An ultrasonic diagnostic apparatus has a function of measuring ultrasonic tomographic images (B-mode images) of a subject. Moreover, the ultrasonic diagnostic apparatus also has a function of measuring time-varying images (M mode) representing the motion of the cardiac wall when measuring the tomographic images of the heart, for example. Furthermore, the ultrasonic diagnostic apparatus is not limited to these functions but also has a function of generating elastic images based on the measurement data of the tomographic images.

In order to realize these plural functions, a technique in which a plurality of measurement modes appropriate for the respective functions is executed in parallel, and in order to generate ultrasonic images from reflected echo signals measured so as to correspond to the respective functions and display the ultrasonic images on a display, a plurality of data conversion sections called digital scan converters (DSC) is provided to generate ultrasonic image data corresponding to the respective functions is proposed (for example, see PTL 1).

### CITATION LIST

### Patent Literature

[PTL 1] JP-B-3612358

### SUMMARY OF INVENTION

### Technical Problem

However, PTL 1 does not describe the harmonization of processing cycles between different data processes and adjustment of the processing time. That is, since the combination of the processing time between a series of different data processes changes variously depending on the content of different data processes or the data process order, it is necessary to harmonize the processing cycles and adjust the processing time using different methods for each combination of the series of different data processes.

That is, for example, in a data conversion section such as a DSC, there is known the fact that a RF data process of converting RF data (or RF frame data) obtained by digitalizing reflected echo signals into RF data appropriate for reconstructing ultrasonic images and a presentation image data process of converting the RF data into ultrasonic image data appropriate for displaying them on a display have data processing amounts and processing cycles, which change in accordance with an ultrasonic measurement mode.

Therefore, if the RF data process and the presentation image process are performed by separate hardware, since they have different processing cycles, there is a problem in that the processing cycles need to be harmonized between the separate hardware, and the processing time needs to be adjusted.

In order to cope with such a problem, for example, a cine memory having a capacity of storing a plurality of RF data during the RF data process and the presentation image data process is provided so that the processes can be performed by allowing the RF data process side to write processing results in a cine memory without considering its processing time while allowing the presentation image data process side to read the processing results of the RF data from the cine memory at any time. However, if the processing cycles of a series of different data processes are harmonized or the processing time is adjusted using such a structure, a plurality of large-capacity memories such as a cine memory needs to be provided for data processes having different processing cycles. Thus, there is a problem in that the size of the memory has to be increased.

Such a problem is a problem which is not limited to a case where a series of different data processes are executed by different hardware but may occur in a case where the processes are executed by different software threads. Moreover, such a problem is not limited to the exemplary combination of the RF data process of converting RF data into the RF data appropriate for reconstructing ultrasonic images and the presentation image process of converting the RF data into ultrasonic image data appropriate for displaying the RF data on a display but similarly occurs in a case where a plurality of ultrasonic measurement modes which requires related data processes having different processing cycles is executed in parallel.

An object to be solved by the invention is to obviate the need to harmonize the processing cycles between different processors and adjust the processing time when processing a plurality of series of data processes with different processors.

### SOLUTION TO PROBLEM

In order to attain the object, the invention provides an ultrasonic diagnostic apparatus including: an ultrasonic probe configured to transmit and receive an ultrasonic wave to and from a subject; a data conversion section configured to generate ultrasonic image data using RF data based on reflected echo signals received from the subject; and a display configured to display ultrasonic images based on the ultrasonic image data generated by the data conversion section.

Particularly, the data conversion section that generates ultrasonic image data using RF data based on reflected echo signals received from the subject includes a plurality of processors that performs a series of data processes related to an ultrasonic measurement mode and a buffer memory that stores the processing results, and the respective processors perform processes that are assigned to them in accordance with a write request of their own processors, write the processing results in the buffer memory, and read the processing results written in the buffer memory in accordance with a read request of other processors.

Specifically, the data conversion section is formed of a multiprocessor which includes a memory that stores the input RF data, a plurality of processors, a control processor that controls the plurality of processors, a buffer memory, and an internal bus that connects these components to each other. The buffer memory includes a plurality of internal buffers set so as to correspond to the respective processors. The respective internal buffers are formed to have a plurality of memory areas. The control processor controls a series of data processes related to the ultrasonic measurement mode to be distributed to and executed by the plurality of processors based on the RF data stored in the memory and assigns the respective internal buffers so as to correspond to the respective processors. The respective processors designate one of the plurality of memory areas in the internal buffer, to which they are assigned, in accordance with a write request to write the processing results of their own processor and cause the processing results to be written to the designated memory area. After a write disable request of their own processor is input, the respective processors permit to read the processing results written in the memory area in accordance with a read request of other processors.

In this way, a buffer memory having a plurality of internal buffers set so as to correspond to respective processors is provided, and the respective internal buffers are divided into a plurality of memory areas. Thus, the processing results of the respective processors executing a series of data processes in a distributed manner can be written to one of the plurality of memory areas corresponding to the upstream-side processor at any time regardless of the processing cycle and the processing time of the downstream-side processor of the series of data processes. Moreover, the downstream-side processor can read the processing results of the upstream-side processor from the memory area in which the processing results are written at any time and execute its process regardless of the processing cycle and the processing time of the upstream-side processor. In this way, when a series of plural data processes are distributed and processed by different processors, it is possible to obviate the need to harmonize the processing cycles between these processors and adjust the processing time.

In this case, when there is a write request to write the processing results of their own processor, it is preferable that the respective processors designate a memory area other than the memory area in which reading of other processors is continuously performed and cause the processing results to be written to the designated memory area. That is, when the processing cycle of a downstream-side processor of a series of processes is long, and the processing cycle of an upstream-side processor is short, a write request to write the next processing results of the upstream-side processor may be output during the period when the reading of the downstream-side processor is not completed. In this case, by designating a memory area other than the memory area in which data are being read and writing data in the designated memory area, it is possible to obviate the need to harmonize the processing cycles between processors and adjust the processing time. In this case, three or more memory areas need to be set so as to correspond to the respective processors.

Conversely, when the processing cycle of the upstream-side processor is long, and the processing cycle of the downstream-side processor is short, the content of one memory area in which the processing results of the upstream-side processor are written can be continuously read twice and used. However, when a write request to write the processing results of the upstream-side processor is output during the reading, a memory area other than the memory area in which data are being read is designated and data are written in the designated memory area similarly to the above.

As an exceptional case of the invention, if the processing cycles of the upstream-side processor and the downstream-side processor are the same, there maybe two memory areas corresponding to the upstream-side processor. An example of such a case is a case in which, for example, a RF data processing step is distributed to and executed by two processors. However, in this case, if the processing cycle of the downstream-side processor is different from the processing cycle of a further downstream-side processor, since it is necessary to set three or more memory areas, this does not depart from the spirit of the invention.

In the invention, when there are many memory areas in which the processing results of their own processors are written, it is preferable that the respective processors designate a memory area in which the latest processing results are written and outputs a read permission in accordance with a read request of other processors. In this way, since the downstream-side processor can execute a data process based on the latest processing results, is possible to secure real-time properties.

In the invention, the ultrasonic diagnostic apparatus may further include a controller that controls the beam forming section and the transmission/reception switching section, the controller may add a measurement mode code indicating the ultrasonic measurement mode input from an input section to the reflected echo signals and transfer the reflected echo signals to the memory of the data conversion section, and the control processor may distribute the series of data processes to a plurality of processors based on the measurement mode code added to the reflected echo signal and stored in the memory and cause the data processes to be performed by the plurality of processors. In this way, a data conversion process can be automatically executed in accordance with the ultrasonic measurement mode.

### ADVANTAGEOUS EFFECTS OF INVENTION

According to the invention, it is possible to obviate the need to harmonize the processing cycles between different processors and adjust the processing time when processing a plurality of series of data processes with different processors.

### BRIEF DESCRIPTION OF DRAWINGS

FIG. 1 is a block configuration diagram of one embodiment of an ultrasonic diagnostic apparatus of the invention.

FIG. 2 is a flowchart of a processing operation of a control processor in the embodiment shown in FIG. 1.

FIG. 3 is a flowchart of a first embodiment wherein a B-mode image process is executed by two processors using the embodiment shown in FIG. 1.

FIG. 4 is an example of an operation time chart of the first embodiment shown in FIG. 3.

FIG. 5 is another example of an operation time chart of the first embodiment shown in FIG. 3.

FIG. 6 is a block configuration diagram of a modification of the embodiment shown in FIG. 1.

FIG. 7 is a flowchart of a second embodiment wherein a B-mode image process and a D-mode measurement process are executed by four processors using the embodiment shown in FIG. 1.

### DESCRIPTION OF EMBODIMENTS

Hereinafter, embodiments of an ultrasonic diagnostic apparatus to which the invention is applied will be described. In the following description, components having the same functions will be denoted by the same reference numerals, and redundant description thereof will be omitted.

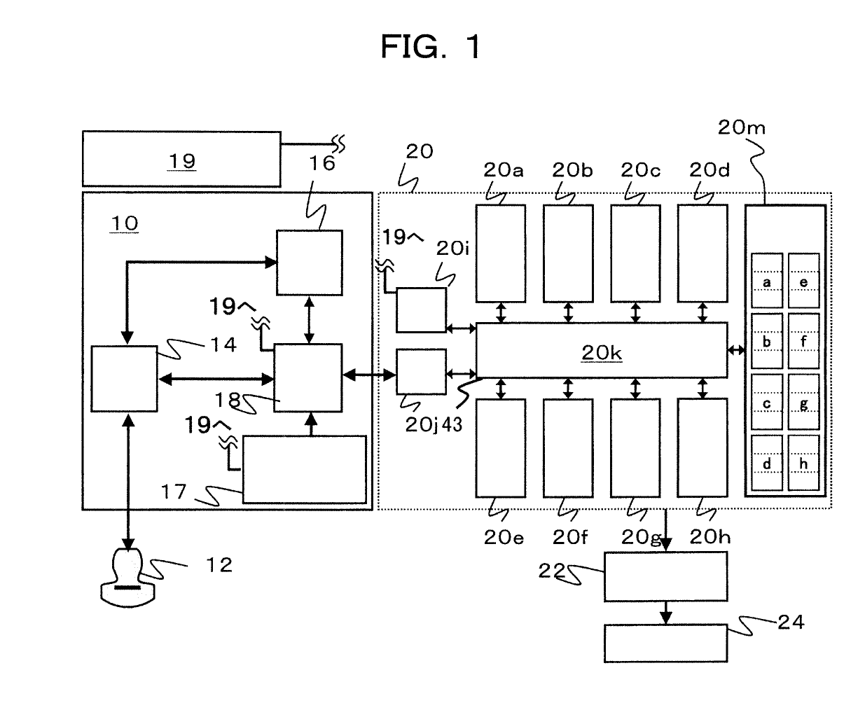

FIG. 1 is a block configuration diagram of one embodiment of an ultrasonic diagnostic apparatus of the invention. As shown in FIG. 1, an ultrasonic diagnostic apparatus of the invention is configured to include an ultrasonic measurement section 10, a data conversion section 20 that performs a series of predetermined data processes set for each ultrasonic measurement mode on RF data which are ultrasonic measurement data measured by the ultrasonic measurement section 10 to thereby generate ultrasonic image data, a video memory 22 that stores ultrasonic image data converted by the data conversion section 20, a display 24 that displays the ultrasonic images and the like stored in the video memory 22, and other components.

The ultrasonic measurement section 10 has configurations necessary for performing well-known ultrasonic measurement, and in the example shown in the figure, is configured to include an ultrasonic probe 12 (PROBE) that transmits an ultrasonic beam to a subject and receives reflected echo signals from the subject, a transmission/reception switching section 14 (PRB) that switches transmission/reception of the ultrasonic probe 12, and a beam forming section 16 (DBF) that supplies a signal for transmitting the ultrasonic beam to the ultrasonic probe 12. The ultrasonic measurement section 10 also includes a setting section 17 (CONSOLE) that sets ultrasonic measurement modes and the like, and a controller 18 (CONT) that controls the transmission/reception switching section 14 and the beam forming section 16 in accordance with the ultrasonic measurement mode set by the setting section 17. Moreover, the information which is input and set to the respective destinations by the setting section 17 is input to a host computer 19, and the host computer 19 is configured to be capable of controlling the transmission/reception switching section 14 and the beam forming section 16 using the controller 18 in accordance with the ultrasonic measurement mode which is input from the setting section 17. Furthermore, the host computer 19 is configured to be capable of controlling the data conversion section 20 using the controller 18.

The ultrasonic probe 12 (PROBE) has vibrators which are arranged in the long-axis direction of the ultrasonic probe so as to correspond to the first to m-th channels. Here, when the vibrators are divided into k channels in the short-axis direction and arranged so as to correspond to the first to k-th channels, by changing the delay time applied to the respective vibrators (first to k-th channels) in the short-axis direction, the transmission and reception beams can also be focused in the short-axis direction. Moreover, when the amplitude of ultrasonic transmission signals applied to the respective vibrators in the short-axis direction is changed, the transmission weighting is realized. On the other hand, when the amplification or attenuation level of ultrasonic reception signals from the respective vibrators in the short-axis direction is changed, the reception weighting is realized. Furthermore, when each of the vibrators in the short-axis direction is turned ON and OFF, an aperture can be controlled.

The ultrasonic probe 12 may have vibrators which are formed of a piezoelectric element and may have vibrators which are formed of a semiconductor called a CMUT (Capacitive Micromachined Ultrasonic Transducer: see, for example, IEEE Trans. Ultrason. Ferroelect. Freq. Contr. Vol. 45, pp. 678-690, May 1998).

The transmission/reception switching section 14 performs the function of an interface that supplies a transmission signal to the ultrasonic probe 12 and processes the received reflected echo signals to generate digitalized RF data. That is, the transmission/reception switching section 14 has a function of performing a reception process such as amplification, A/D conversion, a process of aligning the phases between a plurality of vibrators and adding the phase signals, and the like on the received reflected echo signals to thereby generate digitalized RF data. Moreover, the transmission/reception switching section 14 has the function of a reception circuit that receives the reflected echo signals from the internal part of the subject in response to an ultrasonic beam transmitted to the subject and collects biological information.

The beam forming section 16 is a transmission circuit that controls the ultrasonic probe 12 to output an ultrasonic beam, and is configured to control the transmission time of ultrasonic pulses for driving a plurality of vibrators of the ultrasonic probe 12 to form an ultrasonic beam toward a focal point set within the subject. Moreover, the beam forming section 16 is configured to electronically scan the ultrasonic beam in the arrangement direction of the vibrators of the ultrasonic probe.

The setting section 17 allows an operator to input various parameters such as a desired ultrasonic measurement mode, patient information, and a capturing position using a keyboard and a trackball on a console.

The controller 18 is a control computer system for controlling the transmission/reception switching section 14, the beam forming section 16, and the data conversion section 20 to function appropriately based on various parameters input by the setting section 17. Moreover, the controller 18 is configured to transfer the RF data output from the transmission/reception switching section 14 to the data conversion section 20. At this time, a measurement mode code indicating the ultrasonic measurement mode input from the setting section 17 is associated with the RF data and transferred to the data conversion section 20.

The data conversion section 20 is formed to include a plurality of processors 20a to 20h, a control processor 20i that collectively controls the processors 20a to 20h, a memory 20j (MEMORY) that stores the RF data transmitted from the controller 18, and a buffer memory 20m. Moreover, the data conversion section 20 also includes an internal bus 20k that is capable of communicating data with the processors 20a to 20h, the control processor 20i, the memory 20j, and the buffer memory 20m.

The buffer memory 20m is formed to include a plurality of internal buffers a to h which is set so as to correspond to each of the plurality of processors 20a to 20h. The respective internal buffers a to h are formed to have a plurality of memory areas (in the example shown in the figure, three memory areas). However, the number of memory areas is not limited to this, and it is sufficient to have at least two memory areas.

The control processor 20i controls the processors 20a to 20h connected through the internal bus 20k. That is, the control processor 20i controls the processors 20a to 20h so that a series of data processes associated with an ultrasonic measurement mode are distributed to one or plural of the processors 20a to 20h and performed on the RF data stored in the memory 20j. For example, the control processor 20i analyzes the measurement mode code associated with the RF data stored in the memory 20j and assigns processing programs of a series of data processes related to the ultrasonic measurement mode to one or plural of the processors 20a to 20h. Moreover, the respective internal buffers a to h are assigned so as to correspond to the respective processors 20a to 20h.

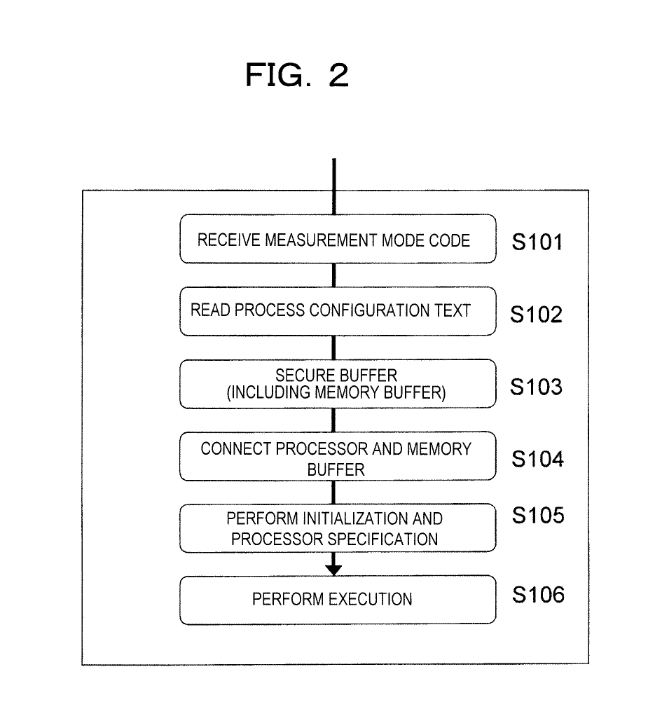

FIG. 2 shows a processing flowchart of the control processor 20i. The control processor 20i receives the measurement mode code which is associated with the RF data and stored in the memory 20j from the controller 18 (S101) and reads a process configuration text corresponding to the measurement mode code from the buffer memory 20m (S102). In the process configuration text, a plurality of processing programs is set so as to correspond to various ultrasonic measurement modes, and the order of determining the respective processors 20a to 20h to which execution of these processing programs is assigned is set. Moreover, in accordance with the setting of the process configuration text, a required buffer memory including the internal buffers a to h is secured (S103). Subsequently, the secured internal buffers a to h are associated with the respective processors 20a to 20h (S104). Moreover, the processing programs are assigned to the respective processors 20a to 20h to perform initialization (S105), and the processes are executed (S106).

The upstream processor 20a to 20h on the uppermost stream side of the series of data processes read the RF data stored in the memory 20j in accordance with an assigned processing program and perform a data process. Based on the processing results, the downstream processors 20a to 20h on the downstream side execute a data process in accordance with the assigned processing program, and the downstream processor 20a to 20h on the lowermost side generate ultrasonic image data and output the same to the video memory 22.

As the ultrasonic measurement mode, various ultrasonic measurement modes such as an A-mode image, a B-mode image, a color flow mapping (C)-mode image, a Doppler (D)-mode image, an elastic (E)-mode image, and an M-mode image are widely known. Moreover, an ultrasonic measurement mode wherein 3D ultrasonic images are reconstructed based on a plurality of B-mode images measured continuously along the body surface of a subject is also known.

The data conversion section 20 may use a multiprocessor, for example, CELL, which is a processor standardized by an architecture in which a plurality of processor cores is integrated in a single package. The CELL is an abbreviation of Cell Broadband Engine (registered trademark) and is a microprocessor developed by Sony Computer Entertainment Corporation and the like. When using CELL, the processors 20a to 20h are configured as SPE (Synergistic Processor Element), the control controller 20i is configured as PPE (PowerPC Processor Element), and the internal bus 20k is configured as EIB (Element Interconnect Bus).

The video memory 22 stores the ultrasonic images formed by the data conversion section 20 by combining the same with the character or graphic information such as patient information or body mark information, and the graphic information set by the setting section 17. The controller 18 also has the function of a display controller that selects and controls the display format to be used for displaying images.

The display 24 displays the ultrasonic images stored in the video memory 22, and is formed, for example, of a CRT monitor or a liquid crystal monitor. The display 24 may only need to be capable of displaying ultrasonic images and display images from which an operator can make a diagnosis, and the invention can be applied to any display regardless of whether it outputs analog signals or digital signals.

In ultrasonic diagnostic apparatus, there is a demand to decrease the circuit size for a data process contributing to miniaturization of the system and a demand to increase the data processing speed contributing to performance of the system. There is still a technical problem that the two demands are in a trade-off relationship. However, the use of a single-package multiprocessor of the present embodiment can contribute to decreasing the circuit size and increasing the data processing speed. Moreover, although the number of processors 20a to 20h is eight as an example in FIG. 1, since the number can be optionally set in accordance with the processing capability of the processor, the number may be any natural number.

Hereinafter, the detailed configuration and operation of the data conversion section 20 of the ultrasonic diagnostic apparatus shown in FIG. 1 will be described based on embodiments of a series of data processes related to specific ultrasonic measurement modes.

### FIRST EMBODIMENT

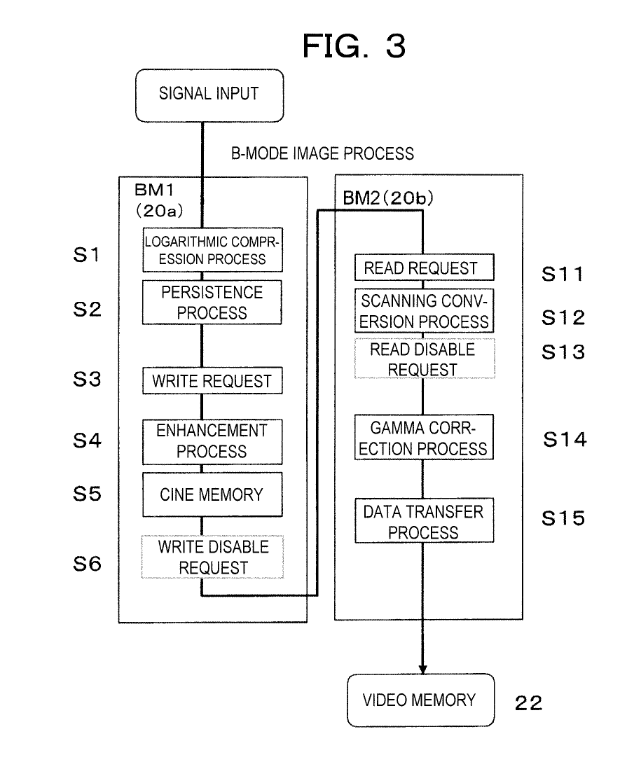

FIG. 3 shows a flowchart of a data process in the data conversion section 20 that visualizes B-mode images which are representative examples of the ultrasonic measurement mode. In this embodiment, it will be described that in order to speed up a B-mode image process, a series of data processes are performed by assigning the processes to two processors 20a and 20b. That is, an RF frame data process of converting RF frame data which are measured by the ultrasonic measurement section 10 and stored in the memory 20j into RF frame data appropriate for reconstructing B-mode images and a presentation image data process of converting the RF frame data into B-mode image data appropriate for displaying them on the display have different processing cycles.

Therefore, the control processor 20i analyzes the measurement mode code stored in the memory 20j to be added to the RF frame data to recognize that a process of reconstructing B-mode images is to be performed. Moreover, in order to perform specific processes up to generation of the B-mode images by a pipeline method, as shown in FIG. 3, the control processor 20i assigns an RF frame data process BM1 to the processor 20a and a presentation image data process BM2 to the processor 20b. Moreover, the control processor 20i sets the processor 20a so as to write the processing results of the process BM1 in the internal buffer a and sets the processor 20b so as to read the processing results written in the internal buffer a and perform the process BM2. Moreover, the control processor 20i sets the processor 20b so as to output the processing results of the process BM2 to the video memory 22.

As shown in FIG. 3, the series of data processes necessary to generation of B-mode images includes the RF frame data process BM1 which includes a logarithmic compression process S1, a persistence process S2, and an enhancement process S4 and the presentation image data process BM2 which includes a scanning conversion process S12, a gamma correction process S14, and a data transfer process S15. The logarithmic compression process S1 involves compressing the dynamic range of RF frame data, which is 2 to the 20th power, for example, to a relatively small dynamic range on the circuit. In the case of the present embodiment, the dynamic range is compressed to a dynamic range of the display 24. The persistence process S2 involves averaging RF frame data which have been subjected to the logarithmic compression process S1 and are displayed on the same pixels on the display 24.

The enhancement process S4 involves enhancing the edges of the RF frame data which have been subjected to the persistence process S2 so that the boundaries between the pixels become definite.

The scanning conversion process S12 involves converting the coordinates of the pixels having been subjected to the enhancement process from the scanning coordinates of the ultrasonic beam to the scanning coordinates on a display monitor. The gamma correction process S14 involves correcting the display gradation of the pixels having been subjected to the scanning conversion process S12 based on a gamma curve that determines the domain of definition of the pixels and the codomain. The data transfer process S15 involves transferring images (B-mode images) obtained through the gamma correction process S14 to the video memory 22.

Here, in the RF frame data process BM1, the data processes are executed at the frame rate of the RF frame data. On the other hand, in the presentation image data process BM2, the data processes are executed at the display refresh cycle (video rate) of the display 24. Since the frame rate and the video rate are generally different from each other, these data processes (refresh) are performed at different times.

Here, when a series of plural data processes are distributed and processed by different processors using the buffer memory 20m which is a characteristic portion of the present embodiment, a method of obviating the need to harmonize the processing cycles between these processors and adjust the processing time will be described. That is, the reason why the presentation image data process BM2 can always output and display the latest ultrasonic image data to the display 24 through the video memory 22 without performing a standby process for realizing synchronization between the RF frame data process BM1 and the presentation image data process BM2 will be described.

The buffer memory 20m of the present embodiment has the internal buffers a to h so as to correspond to each of the respective processors 20a to 20h, and the respective internal buffers a to h are formed to have plural (three in the present embodiment) memory areas having the same size. In the present embodiment, the use state of the three memory areas is managed by the respective processors 20a to 20h. Here, the use state of the three memory areas can be classified into three states which are “read ready memory” Mready, “read designated memory” Mread, and “write designated memory” Mwrite. When the number of memory areas is Mnum (3 in the present embodiment), the internal memories of the respective internal buffers a to h are referred to as “0”, “1”, . . . , “Mnum-1”, which indicate the starting addresses of the respective memory areas, respectively. Here, a memory in the read ready state can be described as a memory “Mready”. For example, if “read ready memory” is Mready=0, it means that a memory area “0” among the Mnum memory areas is in the read ready state. Moreover, if Mread=1 and Mwrite=2, it means that a memory area “1” is in the read designated state and a memory area “2” is in the write designated state. Moreover, in the initial state of the respective memory areas, all memory areas are initialized to “−1” indicating that they are in the unavailable state.

As shown in FIG. 3, when “write request” is output from the processor 20a in the process S3 of the RF frame data process BM1, the processor 20a determines “write designated memory” Mwrite of a write destination based on Equation (1).

Mwrite=(Mwrite+1)% Mnum (1)

Here, “%” in Equation (1) is an operator indicating a remainder.

If Mwrite calculated in Equation (1) is “read designated memory” Mread, it means that the presentation image data process BM2 is presently reading the content of the memory area. Thus, the presentation image data process BM2 will perform a wrong process if it performs writing on the memory area during the reading. Therefore, it is not possible to designate the memory area Mread as “write designated memory”. In this case, Equation (1) is computed again based on the calculated Mwrite, the address of the calculated memory area Mwrite is designated and sent back to the requesting processor 20a.

In this way, the processor 20a stores the processing results in the memory area of the designated address upon completion of the processing of the enhancement process S4. Moreover, in the present embodiment, after the processing results of the enhancement process S4 are stored in the memory area of the internal buffer a, the same processing results are stored in a cine memory set to the memory 20j in step S5 of FIG. 2. However, the process of step S5 may be omitted. Moreover, when the enhancement process S4 or the write process of step S5 ends, the processor 20a outputs “write disable request” to the control processor 20i in step S6.

When “write disable request” is input, the processor 20a changes the state of the memory area in which the write process has ended to “read ready memory” Mready in accordance with Equation (2) below.

Mready=Mwrite (2)

On the other hand, when “read request” is input from the processor 20b in step S11 of the presentation image data process BM2, the processor 20a changes the “read ready memory” Mready state to “read designated memory” Mread in accordance with Equation (3) below to designate the address and send it back to the requesting processor 20b.

Mread=Mready (3)

In response to this, the processor 20b reads the latest processing results from the memory area of the designated address and executes the scanning conversion process S12. Moreover, when the scanning conversion process S12 ends, the processor 20b outputs “read disable request” to the processor 20a. In response to this, the processor 20a initializes the “read designated memory” Mread in accordance with Equation (4) below.

Mread=−1 (4)

By managing the internal buffer a in such a manner, the respective processors 20a can continuously write the latest processing results alternately in two different memory areas during the period when a read process is performed by other processors. Moreover, when a new read process occurs, the latest content of the memory area which is updated at the last time is read.

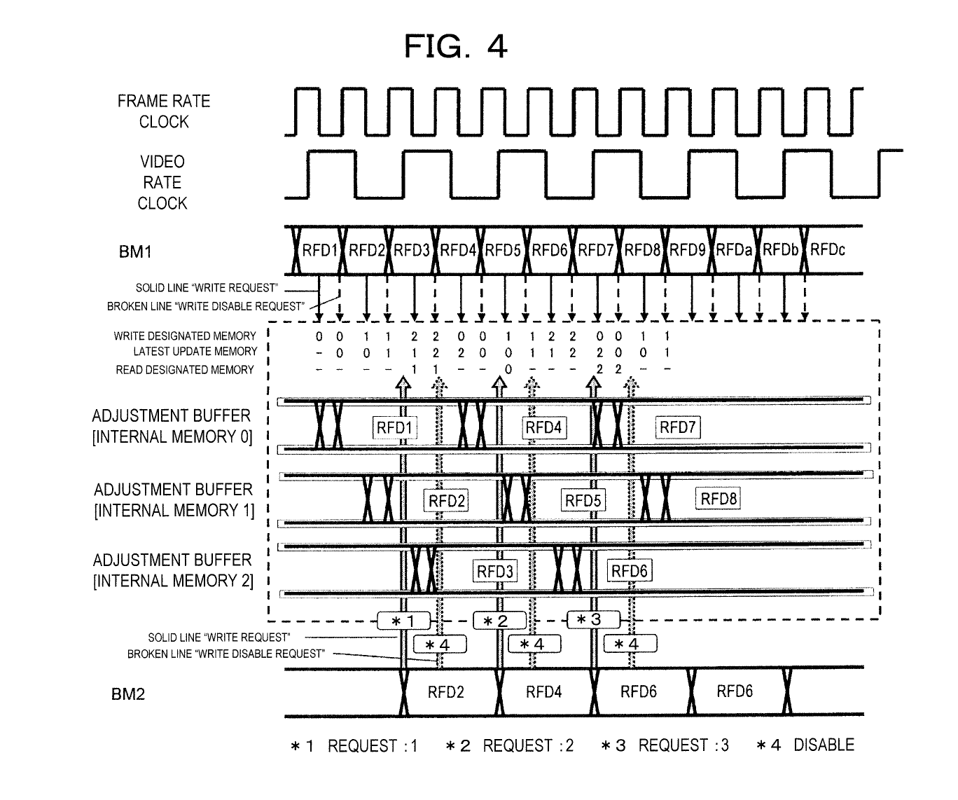

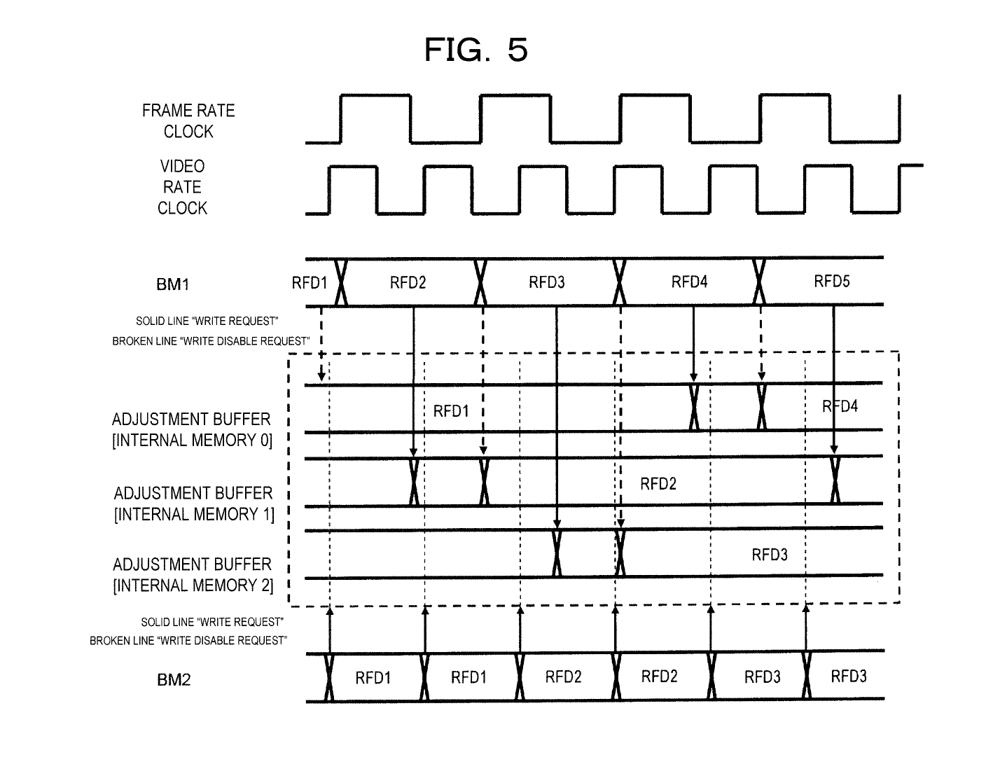

The operation related to the RF frame data process BM1 and the presentation image data process BM2 of the first embodiment will be described using specific time chart examples shown in FIGS. 4 and 5. FIG. 4 shows the execution cycles of the data processes when the frame rate clock which is the operation cycle of the RF frame data process BM1 is faster than the video rate clock which is the operation cycle of the presentation image data process BM2. On the other hand, FIG. 5 shows the execution cycles of the data processes when the frame rate clock which is the operation cycle of the RF frame data process BM1 is slower than the video rate clock which is the operation cycle of the presentation image data process BM2.

As shown in FIG. 4, the RF frame data process BM1 executed by the processor 20a outputs a write request when the logarithmic compression process S1 and the persistence process S2 are completed for the RF data RF1 which are the first processing target (step S3). In this way, the address of “write designated memory” is designated by the function of the processor 20a managing the internal buffer a. Subsequently, the enhancement process S4 is performed, and the RF data RFD1 which are the processing results are stored in the designated memory area. When the enhancement process S4 ends, the RF data stored in the memory area are stored in the cine memory (S5). Moreover, “write disable request” is output. In response to this, the function of the processor 20a managing the internal buffer a changes the state of the memory area to which data have been presently written from the write designated memory area state “2” to the “read ready memory” state “0”. Moreover, the RF frame data process BM1 executes a process on RF data which are the next processing target and stores RF data RFD2 which are the processing results in a memory area of which the address is designated as the “write designated memory” state “2” rather than the “read ready memory” state “0”. That is, the RF data RFD1 and the RF data RFD2 are written in different memory areas. Similarly, frame data processes are sequentially executed in accordance with the frame rate, and the processing results are written in designated memory areas.

On the other hand, the presentation image data process BM2 of the processor 20b operates in a video rate sufficiently lower than the frame rate of the process BM1, and the process on the RF data RFD2 is completed before the presentation image data process BM2 starts the process based on the RF data RFD1. When “read request” occurs at the start time of the presentation image data process BM2 (S11), the processor 20a designates a memory area having the state “1” in which the latest RF data RFD2 of the processor 20b are stored and permits a read process. In this way, the presentation image data process BM2 performs the scanning conversion process, the gamma correction process, and the data transfer process on the acquired data RFD2 and outputs B-mode image data to the video memory 22. That is, the presentation image data process BM2 processes the data RF2 without processing the data RF1. Moreover, the memory area designated by “read request” is not overwritten by the process BM1 of the processor 20a until “read disable request” is issued.

As shown in FIG. 4, when the frame rate which is the operation cycle of the process BM1 is faster than the video rate which is the operation cycle of the process BM2, and it is unable to display all processed data on the display, it is possible to display only the latest acquired data without overwriting images and to display ultrasonic images without any time-phase delay. In the description above, although the memory area is designated by the addresses of the respective memory areas, the invention is not limited to this, and a link list of the handles or instance names of the respective memory areas may be created and used instead of the addresses.

As shown in FIG. 3, the RF frame data process BM1 executed by the processor 20a outputs a write request when the logarithmic compression process S1 and the persistence process S2 are completed for the RF data RF1 which are the first processing target (S3), and stores the RF data RFD1 which are the processing results of the enhancement process S4 in a memory area designated by the address of “write designated memory area”. When the enhancement process S4 ends, the RF data stored in the memory area are stored in the cine memory (S5) and “write disable request” is output.

The process BM2 of the processor 20b operates in a video rate that is sufficiently faster than the frame rate which is the operation cycle of the process BM1 of the processor 20a. Therefore, the process BM1 of the processor 20a outputs “read request” plural times to the processor 20a before the processes of the process BM2 of the processor 20b end. At this time, the processor 20a sets “latest updated buffer” so that a memory area in which the data RF1 are stored is in the state “0” in accordance with the “write disable request” issued when the process on the RF data RF1 is completed.

Therefore, in accordance with the “write request” which the process BM1 of the processor 20a issues in order to store the processing results of the data RF2, a read destination memory area indicates a memory area different from the memory area in which the data RF1 are stored. In this way, even when the process BM2 of the processor 20b outputs “read request” plural times, the content of the memory area being processed by the process BM1 of the processor 20a will not be read. That is, when the frame rate which is the operation cycle of the process BM1 is slower than the video rate of the operation cycle of the process BM2, and it is necessary for the display to display the same data plural times, it is possible to display only the latest acquired data which have been processed last without displaying the images being processed.

Moreover, as described above, although the RF frame data process BM1 operates at the frame rate, if the scanning range of the ultrasonic wave is narrow, RF frame data are collected from a subject at a speed which exceeds the performance of the display 24 and the visibility limit of the human eyes. Moreover, if the scanning range of the ultrasonic wave is wide, the frame rate decreases too much. In the present embodiment, since the RF frame data are stored temporarily in the cine memory in step S5, all RF frame data acquired from the subject can be reproduced again slowly after the process on the RF frame data ends. Moreover, it is possible to observe the details of lesions which were not viewable.

In the embodiment shown in FIG. 1, although the content of the data processes assigned to the processors 20a to 20h can be stored in the buffer memory 20m as “process configuration text,” the invention is not limited to this, the “process configuration text” may be stored in the memory 20j. In the list of these data processes, the processing order and the processing content, for example, shown in FIG. 2 are included, and the configuration of a processor that transfers data from the process BM1 to the process BM2 using two processors 20a and 20b is included. This information is independent for each diagnostic mode of the ultrasonic diagnostic apparatus such as a B mode and a Doppler mode and is selected by the user through the setting section 17, and necessary information is transferred to the controller 18 and the control processor 20i in accordance with the input ultrasonic measurement mode. The control processor 20i reads the processing content and the processor configuration corresponding to the ultrasonic measurement mode from the buffer memory 20m and actually assigns processes to the processors 20a to 20h, whereby different functions are realized for each ultrasonic measurement mode of the ultrasonic diagnostic apparatus such as the B mode and the Doppler mode.

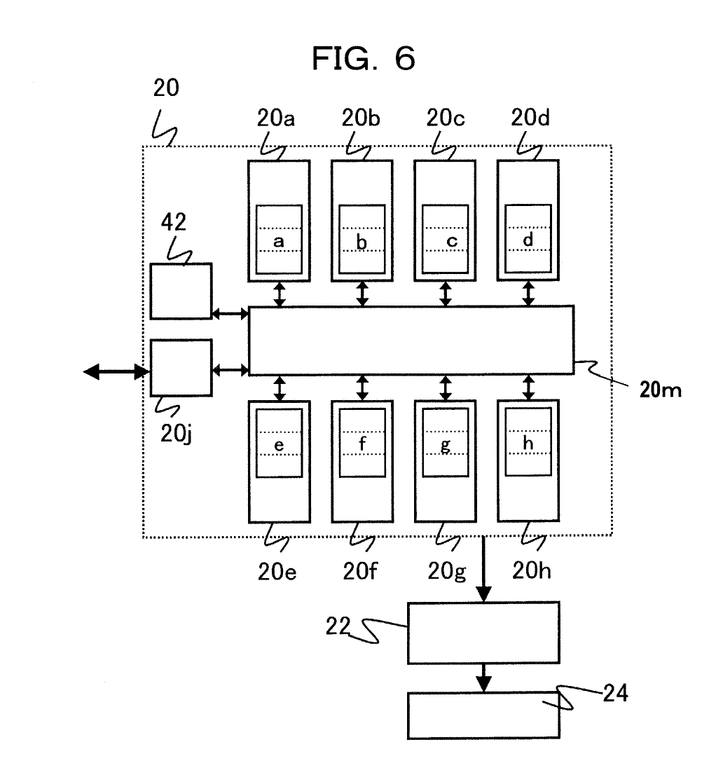

In the embodiment shown in FIG. 1, although the buffer memory 20m is separated from the respective processors 20a to 20h and connected to the internal bus 20k, the invention is not limited to this, and as shown in FIG. 6, the buffer memory 20m may be provided to be integrated into the respective processors 20a to 20h.

### SECOND EMBODIMENT

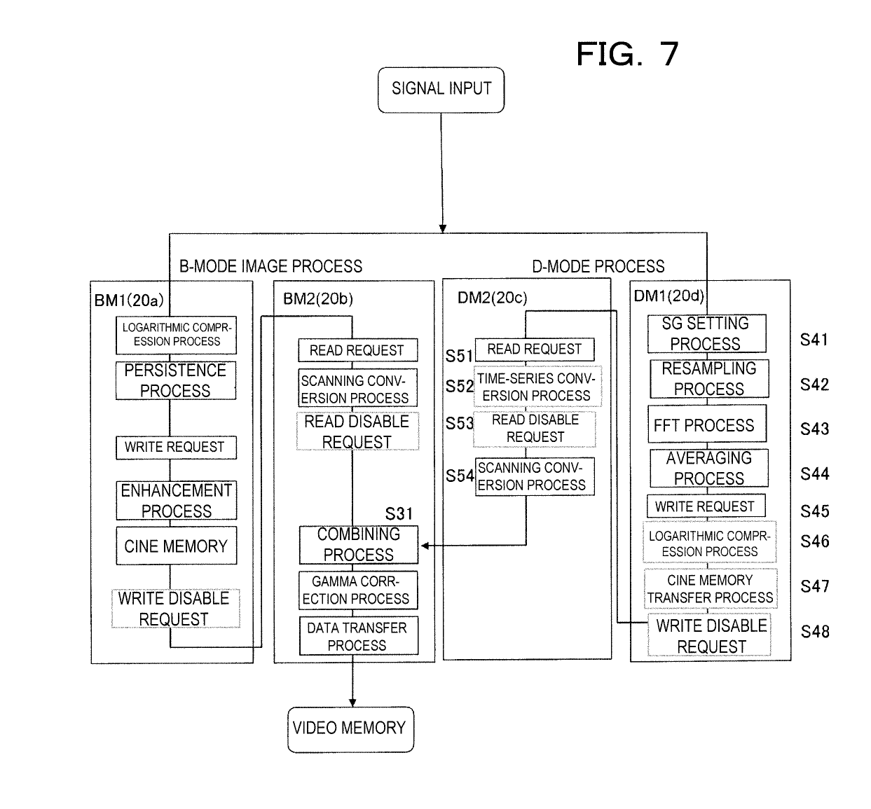

FIG. 7 shows a flowchart of a second embodiment wherein a series of data processes including a data process of visualizing B-mode images of the ultrasonic measurement mode and a Doppler mode (D mode) process of the ultrasonic measurement mode are executed in parallel by the data conversion section 20. In order to execute the present embodiment, four data processes BM1, BM2, DM1, and DM2 are executable in parallel using the processors 20c and 20d in addition to the processors 20a and 20b of the first embodiment. Moreover, as described above, the present embodiment can be realized by mounting four software threads instead of the processors 20a, 20b, 20c, and 20d.

In the present embodiment, switching between an ultrasonic measurement mode for measuring B-mode images and an ultrasonic measurement mode for performing a Doppler process is performed by the user through the setting section 17. The data conversion section 20 receives a Doppler mode code transferred from the controller 18 through the control processor 20i, reads a processing content of the designated Doppler mode and the processor configuration from the buffer memory 20m, and actually assigns processes to the processors 20a to 20d. When the Doppler mode is selected, four processors 20a to 20d are used, and different processing contents of the processes BM1, BM2, DM1, and DM2 are assigned to the respective processors 20a to 20d.

Here, a video rate process thread of the B-mode images as indicated by the process BM2 executed by the processor 20b and a video rate process thread of the Doppler mode as indicated by the process DM2 executed by the processor 20d are operated at the same time to be synchronized with the refresh rate (image refresh cycle) of the video memory. Moreover, the output images of the process DM2 and the output images of the process BM2 are combined by a combination process S31, and the combined images are output to the video memory 22. Here, the process DM1 is a Doppler-mode ultrasonic synchronization process thread which is a thread that performs processing at the cycle in the unit of ultrasonic beams and which has an operation cycle of Lpre (times per one data unit). If the operation cycle of the process DM2 is Lpost (times per one data unit), the internal buffers a to d of the buffer memory 20m need to have number Mnum_dm of memory areas wherein Mnum_dm is Lpre/Lpost. The number Mnum_dm of memory areas of these internal buffers a to h is stored in the memory 20j as process configuration text similarly to the processing content.

In FIG. 7, the processes BM1 and BM2 of the B-mode image process are the same as those of FIG. 3 except for the combining process S31 of the process BM2. The process DM1 of the D-mode process is a well-known data process and includes a sample gate (SG) setting process S41, a resampling process S42, an FFT process S43, an averaging process S44, a logarithmic compression process S46, and a cine memory transfer process S47. A write request S45 is provided before the logarithmic compression process S46, and a write disable request S48 is output after completion of the cine memory transfer process S47.

The process DM2 of the D-mode process includes a time-series conversion process S52 and a scanning conversion process S54, and a read request S51 is output before the start of the time-series conversion process S52, so that the processing results of the logarithmic compression process S46 stored in the memory area of the internal buffer are read. Moreover, when the time-series conversion process S52 ends, a read disable request is output (S53), and the memory area is open. The processing results of the scanning conversion process S54 are combined with the B-mode images in the combining process S31 of the B-mode process BM2 and combined images are generated.

As described above, according to the second embodiment, a buffer memory having a plurality of internal buffers set so as to correspond to respective processors is provided, and the respective internal buffers are divided into a plurality of memory areas. Thus, the processing results of the respective processors executing a series of data processes in a distributed manner can be written to one of the plurality of memory areas corresponding to the upstream-side processor at anytime regardless of the processing cycle and the processing time of the downstream-side processor of the series of data processes. Moreover, the downstream-side processor can read the processing results of the upstream-side processor from the memory area in which the processing results are written at any time and execute its process regardless of the processing cycle and the processing time of the upstream-side processor. In this way, when a series of plural data processes are distributed and processed by different processors, it is possible to obviate the need to harmonize the processing cycles between these processors and adjust the processing time.

### Reference Signs List

10: ULTRASONIC MEASUREMENT SECTION

14: TRANSMISSION/RECEPTION SWITCHING SECTION

16: BEAM FORMING SECTION

17: SETTING SECTION

18: CONTROLLER

19: HOST COMPUTER

20: DATA CONVERSION SECTION

20a to 20h: PROCESSOR

20i: CONTROL PROCESSOR

20j: MEMORY

20m: BUFFER MEMORY

a to h: INTERNAL BUFFER

1. An ultrasonic diagnostic apparatus comprising:
an ultrasonic probe configured to transmit and receive an ultrasonic wave to and from a subject;
a data conversion section configured to generate ultrasonic image data using RF data based on reflected echo signals received from the subject; and
a display configured to display ultrasonic images based on the ultrasonic image data generated by the data conversion section,
wherein the data conversion section includes a plurality of processors that performs a series of data processes related to an ultrasonic measurement mode and a buffer memory that stores the processing results, and
wherein the respective processors perform processes that are assigned to them in accordance with a write request of their own processors, write the processing results in the buffer memory, and read the processing results written in the buffer memory in accordance with a read request of other processors.

2. The ultrasonic diagnostic apparatus according to claim 1,
wherein the data conversion section further includes a memory that stores the input RF data and a control processor that controls the plurality of processors, and
wherein the control processor controls the plurality of processors to perform a series of data processes related to the ultrasonic measurement mode based on the RF data stored in the memory.

3. The ultrasonic diagnostic apparatus according to claim 1,
wherein the buffer memory includes a plurality of internal buffers, and the respective internal buffers are assigned so as to correspond to the respective processors.

4. The ultrasonic diagnostic apparatus according to claim 3,
wherein the respective internal buffers have a plurality of memory areas.

5. The ultrasonic diagnostic apparatus according to claim 4,
wherein the respective processors designate one of the plurality of memory areas in the internal buffer, to which they are assigned, in accordance with a write request to write the processing results of their own processor and cause the processing results to be written to the designated memory area, and
wherein after a write disable request of their own processor is input, the respective processors permit to read the processing results written in the memory area in accordance with a read request of other processors.

6. The ultrasonic diagnostic apparatus according to claim 4,
wherein when there is a write request to write the processing results of their own processor, the respective processors designate a memory area other than the memory area in which reading of other processors is continuously performed and cause the processing results to be written to the designated memory area.

7. The ultrasonic diagnostic apparatus according to claim 4,
wherein when there are a plurality of memory areas in which the processing results of their own processor are written, the respective processors designate a memory area in which the latest processing results are written and output a read permission in accordance with a read request of other processors.

8. The ultrasonic diagnostic apparatus according to claim 1,
wherein the respective processors operate a plurality of data processes in parallel.

9. The ultrasonic diagnostic apparatus according to claim 2, further comprising:
a beam forming section that drives the ultrasonic probe to form a transmission signal for transmitting an ultrasonic beam to the subject; and
a controller that controls a transmission/reception switching section that supplies the transmission signal of the beam forming section to the ultrasonic probe and receives reflected echo signals received by the ultrasonic probe,
wherein the controller adds a measurement mode code indicating the ultrasonic measurement mode input from an input section to the RF data and transfers the RF data to the memory of the data conversion section, and
wherein the control processor distributes the series of data processes to a plurality of processors based on the measurement mode code added to the reflected echo signal and stored in the memory and causes the data processes to be performed by the plurality of processors.

10. A method for generating ultrasonic images comprising:
a step of generating ultrasonic image data using RF data based on reflected echo signals; and
a step of displaying ultrasonic images based on the ultrasonic image data,
wherein the step of generating the ultrasonic image data includes:
a step wherein a plurality of processors that performs a series of data processes related to an ultrasonic measurement mode performs processes that are assigned to them in accordance with a write request of their own processors;
a step of writing the processing results in a buffer memory; and
a step of reading the processing results written in the buffer memory in accordance with a read request of other processors.
